(19) United States
(12) Patent Application Publication
(10) Pub. No.: US 2023/0302203 A1
(43) Pub. Date: Sep. 28, 2023

(54) IMPLANT USING NANOCOMPOSITE HYDROGEL

(71) Applicants: JBM Incorporation, Tokyo (JP); GN Corporation Co Ltd, Yamanashi (JP)

(72) Inventors: Samuel JK ABRAHAM, Yamanashi (JP); Kazutoshi HARAGUCHI, Chiba (JP)

(21) Appl. No.: 18/007,218

(22) PCT Filed: Jul. 30, 2021
(86) PCT No.: PCT/JP2021/028245

§ 371 Date: Jan. 27, 2023

(30) Foreign Application Priority Data
Jul. 30, 2020 (JP) 2020-130868

(51) Int. Cl. A61L 27/52; A61L 27/02; A61L 27/16; A61F 2/12

(52) U.S. Cl. CPC A61L 27/52; A61L 27/025; A61L 27/16; A61F 2/12; A61L 2400/12; A61L 2430/04; A61L 2430/34

(57) ABSTRACT

An implant capable of maintaining, even when being implanted in a body for a long period of time, desirable mechanical properties, non-bioabsorbable properties, and/or biocompatibility is provided. The implant contains a nanocomposite hydrogel obtained through formation of an organic-inorganic network structure in which an amide group-containing polymer compound and an inorganic clay nano-sheet are linked.

### CROSS-REFERENCE TO RELATED APPLICATIONS

This application is a national stage application, filed under 35 U.S.C. 371, of international application no. PCT/JP2021/028245, filed Jul. 30, 2021, and claims the benefit of Japanese Application No. 2020-130868, filed Jul. 31, 2020, each application of which is hereby incorporated herein by reference in its entirety.

### TECHNICAL FIELD

The present invention relates to an implant using a nanocomposite hydrogel.

### BACKGROUND ART

In the medical field, various soft implant materials that require flexibility to be used for prosthesis for a soft tissue or used for a heart valve or the like have been studied. Among such soft implant materials, only a few are used in actual clinical practice, and examples thereof include bioabsorbable materials such as autologous fat, sodium hyaluronate (HA), and collagen, and non-bioabsorbable synthetic polymers such as silicone and polyurethane (NPLs 1 and 2). A soft implant material using a bioabsorbable material has relatively excellent biocompatibility, but is eventually absorbed, and therefore, the initial implantation state cannot be stably maintained for a long period of time. On the other hand, a soft implant material using a non-bioabsorbable material is designed on the premise that it is maintained stably for a long period of time. However, for example, it is reported that in an implantation test for about half a year to three years, it is found that it maintains mechanical properties and has biocompatibility, but when it is actually implanted for a long period of time, a soft implant material collapses or a foreign body reaction occurs (NPLs 3 and 4). Therefore, even a soft implant material using a non-bioabsorbable material still has a problem that the initial implantation state cannot be stably maintained for a long period of time. In order to cope with this problem, various surface treatments for a non-bioabsorbable material have been studied, but a sufficient effect has not necessarily been obtained (NPLs 5 and 6). Therefore, a search for a soft implant material capable of stably maintaining good mechanical properties and biocompatibility for a long period of time in a living body is currently an urgent issue.

A polymer hydrogel has excellent water absorbability, permeability, and flexibility because the material can contain a large amount of water or an aqueous solution, and is used in many fields including the medical field. For example, a weakly crosslinked body of sodium polyacrylate known as a super absorbent material (SAP: super absorbent polymer) absorbs water or an aqueous solution several hundred times or more its own weight, and therefore is widely used in paper diapers and sanitary products. A polymer hydrogel containing poly(2-hydroxyethyl methacrylate) as a main component has high transparency and oxygen permeability in addition to an arc shape suitable for use as a lens, and therefore is widely used as a soft contact lens. In addition, many polymer hydrogels are used in the fields of medicine, pharmaceutical, and analysis. For example, a polyacrylamide gel or an agarose gel is used as an electrophoresis gel, a chitosan gel is used as a wound dressing, and a gelatin hydrogel is used as a drug sustained-release carrier. In addition, poly(N-isopropylacrylamide) coated on a polystyrene dish with radiation undergoes a hydrophilic-hydrophobic transition at its lower limit critical consolute temperature (LCST), and therefore is used as a functional cell culture coating.

As a drawback of such a polymer hydrogel, there was a problem that it is mechanically fragile and cannot be molded into an arbitrary shape. That is, a conventional polymer hydrogel had problems that it is easily ruptured by large stress such as stretching, compression, or twisting, it is difficult to mold it into a large shape or a complicated shape and to mold it into a fine surface morphology, etc.

On the other hand, in recent years, a hydrogel having high mechanical properties and easy moldability into various shapes has been developed by constructing a new network structure. For example, a slide-ring hydrogel designed so that a crosslinking point can move at the molecular level (PTLs 1 and 2, and NPL 7), a nanocomposite hydrogel formed of an organic-inorganic network structure using an inorganic clay nanosheet as a super-polyfunctional crosslinking agent (PTLs 3 and 4, and NPL 8), and a double network hydrogel having an interpenetrating network structure (PTLs 5 and 6, and NPL 9) are exemplified. Among these, the nanocomposite hydrogel is known such that the hydrogel can be prepared in high yield and in any shape by an easy method of performing in situ radical polymerization of a water-soluble monomer in water in the presence of an inorganic clay nanosheet (NPL 10), the mechanical properties of the hydrogel are controlled over a wide range by changing the composition or the like (NPL 11), and the hydrogel exhibits excellent functionality such as temperature responsiveness (PTLs 3 and 4, and NPL 12), cell culturability (PTL 7 and NPL 13), and a self-repairing property (NPL 14).

### CITATION LIST

### Patent Literature

PTL 1: JP3475252B
PTL 2: U.S. Pat. No. 6,828,378B2
PTL 3: JP4545984B
PTL 4: U.S. Pat. No. 6,943,206B2
PTL 5: JP4915867B
PTL 6: US2014/0329975
PTL 7: JP5349728B

### Non Patent Literature

NPL 1: Zhao, P., Zhao, W., Zhang, K., Lin, H., Zhang, X., Polymeric injectable fillers for cosmetology: Current status, future trends, and regulatory perspectives. J. Appl. Polym. Sci., 2019, 137, 48515
NPL 2: BD Ratner., 9.21 Polymeric Implants, Polymer Science A Comprehensive Reference., 2012, Volume 9, 397-411
NPL 3: Dana J Lin, Tony T Wong, Gina A Ciavarra, Jonathan K Kazam, Adventures and Misadventures in Plastic Surgery and Soft-Tissue Implants., Radiographics. 2017 37(7), 2145-2163
NPL 4: Byung Ho Shin1, Byung Hwi Kim1, Sujin Kim, Kangwon Lee, Young Bin Choy, Chan Yeong Heo., Silicone breast implant modification review: overcoming capsular contracture., Biomaterials Research, 2018, 22:37
NPL 5: Nikki Castel, Taylor Soon-Sutton, Peter Deptula, Anna Flaherty, Fereydoun Don Parsa., Polyurethane-Coated Breast Implants Revisited: A 30-Year Follow-Up, 2015; 42(2), 186-93
NPL 6: Medical equipment approval number: 22500BZX00460000, Natrelle 410 breast implant package insert, June 2019 (4th edition)
NPL 7: Okumura, Y., Ito, K., The polyrotaxane gel: a topological gel by figure-of-eight cross-links. Adv. Mater. 2001, 13, 485-487
NPL 8: Haraguchi, K., Takehisa, T., Nanocomposite hydrogels: a unique organic-inorganic network structure with extraordinary mechanical, optical, and swelling/de-swelling properties, Adv. Mater. 2002, 14, 1120-1124
NPL 9: Gong, J. P., Katsuyama, Y., Kurokawa T., Osada, Y., Double-network hydrogels with extremely high mechanical strength, Adv. Mater. 2003, 15, 1155-1158
NPL 10: Haraguchi, K., Nanocomposite hydrogels, Curr. Opin. Solid State Mat. Sci. 2007, 11, 47-54
NPL 11: Haraguchi, K., Farnworth, R., Ohbayashi, A., Takehisa, T., Compositional effects on mechanical properties of nanocomposite hydrogels composed of poly(N,N-dimethylacrylamide) and clay, Macromolecules 2003, 36, 5732-5741
NPL 12: Haraguchi, K., Takehisa, T., Fan, S., Effects of clay content on the properties of nanocomposite hydrogels Composed of Poly(N-isopropylacrylamide) and clay, Macromolecules 2002, 35, 10162-10171
NPL 13: Haraguchi, K., Takehisa, T., Ebato, M., Control of cell cultivation and cell sheet detachment on the surface of polymer/clay nanocomposite hydrogels, Biomacromolecules 2006, 7, 3267-3275
NPL 14: Haraguchi, K. Uyama, K. Tanimoto, H., Self-healing in nanocomposite hydrogel, Macromol. Rapid Commun. 2011, 32, 1253-1258

### SUMMARY OF INVENTION

### Technical Problem

An object of the invention is to provide a soft implant material capable of maintaining desirable mechanical properties, non-bioabsorbable properties, and/or biocompatibility, even when it is implanted or placed in a living body for a long period of time.

### Solution to Problem

The present inventors conducted intensive studies to achieve the above object, and as a result, they found that when a nanocomposite hydrogel obtained through formation of an organic-inorganic network structure in which an inorganic clay nanosheet and an amide group-containing polymer compound are linked was used as a soft implant, desirable mechanical properties, non-bioabsorbable properties, and biocompatibility were maintained over a long period of time in a human living body, and therefore, the nanocomposite hydrogel can be applied to various implants, and thus completed the invention.

That is, the invention relates to the following:
[1] An implant, characterized by containing a nanocomposite hydrogel obtained through formation of an organic-inorganic network structure in which an inorganic clay nanosheet and an amide group-containing polymer compound are linked.
[2] The implant according to [1], characterized by being implanted or placed in a human living body.
[3] The implant according to [1] or [2], characterized in that the mass ratio of the inorganic clay nanosheet to the amide group-containing polymer compound is in a range of 0.1 to 1.8.
[4] The implant according to any one of [1] to [3], characterized in that the amide group-containing polymer compound is a polymer obtained by polymerizing one or more polymerizable unsaturated group-containing water-soluble organic monomers having an amide group such as N-alkylacrylamide, N,N-dialkylacrylamide, acrylamide, N-alkylmethacrylamide, N,N-dialkylmethacrylamide, and methacrylamide.
[5] The implant according to any one of [1] to [4], characterized in that the amide group-containing polymer compound dissolves or swells in water at a biological temperature and is capable of forming a nanocomposite hydrogel having optical transparency at a biological temperature.
[6] The implant according to any one of [1] to [5], characterized by being implanted in a load region.
[7] The implant according to any one of [1] to [6], characterized by being used for prosthesis for a soft tissue.
[8] The implant according to [7], characterized by being used for prosthesis for a soft tissue of the face or the breast.
[9] The implant according to any one of [1] to [8], characterized by being able to be cut and/or bonded at a clinical site.

### Advantageous Effects of Invention

The invention can provide an implant material capable of stably maintaining good mechanical properties, biocompatibility, and non-bioabsorbable properties in a living body. The implant of the invention does not adhere to a surrounding tissue or cause a foreign body reaction, and has excellent biocompatibility. Further, the implant of the invention can maintain the initial mechanical properties and non-bioabsorbable properties even when it is implanted for a long period of time, and therefore, the implant does not collapse or elute, and has unprecedented high safety. In addition, the implant of the invention can be easily processed such as cut or bonded in the air, and therefore, tailor-made processing such as shredding at a clinical site can be realized, thereby achieving improvement of aesthetics and curability. Further, the implant of the invention can be sterilized while maintaining its shape and physical properties and also has a function such as optical transparency as well as having tailor-made processability, and therefore can provide unprecedented easy operation for practitioners. Moreover, the implant can be simply and easily produced, and therefore has excellent economic efficiency, and thus can be applied to a subject with a wide range of diseases.

### BRIEF DESCRIPTION OF DRAWINGS

FIG. 1 shows a CT image of a subject suffering from progressive hemifacial atrophy. From the image, it can be seen that the cheekbone of the subject is missing.

FIG. 2 is a photograph showing that an implant implantation planned region, that is, the circumference of the cheek region of the subject was marked.

FIGS. 3A-3D are photographs showing that a nanocomposite hydrogel sterilized by an autoclave (FIGS. 3A and 3B) is shredded at the time of an operation, and the implant pieces are inserted one piece at a time with tweezers multiple times through incision wounds in a lower part of the orbit and a side part of the auricle so as to reconstruct a soft tissue of the cheek region (FIGS. 3C and 3D).

FIGS. 4A and 4B are photographs showing the appearance of the subject one year after the operation. It can be seen that there is a soft tissue-like swelling reconstructed by the implant in the cheek region of the right cheekbone of the subject.

FIGS. 5A-5D are photographs showing the appearance of the subject 7 years after the operation (A and B) and 9 years after the operation (C and D). It can be seen that the shape of the implant implanted in the cheek region of the right cheekbone of the subject does not change.

FIGS. 6A-6C shows MRI images of the subject 7 years after the operation. It shows a coronal image of the implant implantation region (A), a cross-sectional image of the lower part of the implant implantation region (upper jaw part) (B), and a cross-sectional image of the upper part of the implant implantation region (C). From any of the images, it can be seen that no foreign body reaction such as inflammation or capsular contracture has occurred in and around the implant implantation region.

FIG. 7 shows an implant piece taken out from the subject. It can be seen that there is no deposit or vascular invasion in the implant piece.

### DESCRIPTION OF EMBODIMENTS

Unless otherwise defined in the specification, all technical and scientific terms used herein have the same meaning as those commonly understood by those skilled in the art. All patents, applications, published applications, and other publications referenced herein are hereby incorporated by reference in their entirety.

The invention includes an implant, characterized by containing a nanocomposite hydrogel obtained through formation of an organic-inorganic network structure in which an inorganic clay nanosheet and an amide group-containing polymer compound are linked.

In the present disclosure, the “implant” refers to a structure or a device to be implanted or placed in a living body, and shall include, without limitation, implants of all embodiments of the present disclosure such as a soft implant, a hard implant, a final implant, and an implant piece.

In the present disclosure, the “prosthesis” refers to reinforcement of the form or function of at least a part of a living body by an implant, and shall include all types of aesthetic, prophylactic, and/or therapeutic reinforcement.

The implant of the invention can be implanted or placed in any body part without limitation as long as it is a living body. The body part includes, without limitation, head, neck, upper limb, lower limb, and trunk, and includes any organ, body cavity, lumen, etc. existing in these body parts. The organ includes, without limitation, digestive organs such as stomach and intestine, circulatory organs such as heart, respiratory organs such as lung, urinary organs such as bladder, urethra, and urinary duct, reproductive organs such as testis and uterus, external genitalia such as breast, endocrine organs such as thyroid gland, sensory organs such as eyeball and ear, nervous systems such as brain and spinal cord, and motor organs such as bone and muscle. The body cavity includes, without limitation, cranial cavity, thoracic cavity, and abdominal cavity. The lumen includes, without limitation, oral cavity, urethra, and urinary duct. In addition, the body part includes any biological tissue forming a body part, and the biological tissue includes, without limitation, epithelial tissue, connective tissue, muscle tissue, and nerve tissue, and also includes hard tissues such as bone and soft tissues such as muscle, fat, tendon, ligament, and skin.

In one embodiment, the nanocomposite hydrogel used for the implant of the invention has almost no change in mechanical properties even when it is implanted in a living body for a long period of time. Therefore, it can be suitably used as an implant for implantation in a load region such as face, breast, heart, cartilage, or bone (for example, joint, intervertebral bone, or jaw) where a load can be generated constantly against the implant due to the daily activity of the subject.

Typical examples of the implant for implantation in a load region include an implant for prosthesis for a soft tissue (also referred to as “prosthesis”), an artificial blood vessel, a shunt, an intraocular lens, an artificial joint, a plate, a bolt, an artificial spinal plate, a dental implant, a prosthetic valve, a pacemaker, and an implantable sensor. As the implant for prosthesis for a soft tissue, one that supplements a soft tissue of the breast, face, buttock, or the like is preferred. As the implant for prosthesis for the face, one that supplements a soft tissue of the cheek, nose, forehead, temple, or the like is preferred.

In one embodiment, the nanocomposite hydrogel used for the implant of the invention can maintain a certain degree of adhesiveness in an atmospheric state and high slipperiness in a wet state, and therefore can be suitably used as an indwelling implant to be placed in an organ surface, a body cavity, a lumen, or the like. Typical examples of such an indwelling implant include a catheter, a stent, a contact lens, an organ protection sheet, a sheet for preventing adhesion between organs, and a sheet for fixing a graft (for example, a cartilage sheet for implantation, or the like). As an indwelling method, a conventional method in this technical field can be used.

The implant of the invention may be configured such that the nanocomposite hydrogel forms a part or the whole of the implant. The phrase “forms a part of the implant” means that the implant contains the nanocomposite hydrogel, and the phrase “forms the whole of the implant” means that the implant is formed only of the nanocomposite hydrogel. Therefore, in one embodiment, the implant of the invention contains the nanocomposite hydrogel, and in another embodiment, the implant of the invention is formed of the nanocomposite hydrogel. In the embodiment of containing the nanocomposite hydrogel, the implant of the invention can contain any material or component other than the nanocomposite hydrogel.

In this technical field, implants can typically be broadly divided into a hard implant, in which the elastic modulus of the implant body is high, and a soft implant, in which the elastic modulus of the implant body is low. The hard implant is generally used for the purpose of supplementing a hard tissue and has an implant body made of, for example, a metal, a ceramic, or the like, and having a high strength. The soft implant is generally used for the purpose of supplementing a soft tissue and has a soft implant body made of, for example, silicone, polyurethane, or the like.

The implant of the invention may be a hard implant or a soft implant without limitation, and in any implant, it is only necessary that the nanocomposite hydrogel forms a part or the whole of the implant.

In an embodiment of the hard implant, the implant of the invention, for example, can contain the nanocomposite hydrogel as a soft implant material for imparting a portion having a low elastic modulus to a part of the implant, or as a coating material or the like for imparting an arbitrary function such as biocompatibility, non-adhesiveness, or slipperiness to a hard implant body.

In an embodiment of the soft implant, without limitation, the implant of the invention may be formed of, for example, a single material made of a nanocomposite hydrogel, or may be formed of a nanocomposite gel obtained by combining multiple nanocomposite hydrogels having different elastic moduli, or may be formed by combining with another soft implant material other than the nanocomposite hydrogel. As an embodiment of being formed by combining, the nanocomposite hydrogel can be contained as a soft implant material, a coating material, or the like for imparting an arbitrary function such as desirable mechanical properties, biocompatibility, non-adhesiveness, or slipperiness to a soft implant material.

The shape and size of the implant are not particularly limited as long as the implant can be implanted in a living body. The shape may be, for example, a strip shape, a granular shape, or the like without limitation other than an implant shape generally used in this technical field such as a film shape, a spherical shape, a rod shape, a bolt shape, or a hollow (tube) shape. The size may be, for example in the case of a spherical shape, 10 μm to 1 m, 100 μm to 50 cm, 1 mm to 30 cm, or 5 mm to 25 cm, and is preferably 1 mm to 30 cm.

As a method of implanting or placing the implant, a usual method according to the type of implant can be used. In one embodiment, the implanting or placing method includes a method of implanting or placing a final implant in the body of a subject. In the present disclosure, the “final implant” refers to an implant formed of two or more implant pieces. The two or more implant pieces are preferably of the same material, and more preferably formed of the nanocomposite hydrogel. The volume of the implant piece is, without limitation, ½ to 1/100000, preferably ⅓ to 1/1000 of the volume of the final implant. It can be understood that when the volume of a certain implant piece is ⅓, the final implant can be formed using three such implant pieces.

For example, when a missing part is a breast, multiple pieces, for example, 100 implant pieces are inserted sequentially from an incision wound, the final implant having a form (size and shape) according to the subject such as a size and a curve conforming to the missing breast can be molded at a clinical site. The length of the incision wound can be adjusted according to the size and shape of the implant piece, the method of inserting the implant piece, and the like. The size of the implant piece can be selected according to the size of the final implant and the length of the incision wound planned.

The shape of the implant piece of this embodiment can be a spherical shape, a strip shape, a film shape, a granular shape, or the like without limitation. In the case of a spherical shape, a strip shape, or a film shape, it may be inserted with an instrument capable of filling at the implantation site such as tweezers or forceps, and in the case of a granular shape, it can be filled at the implantation site using a syringe, a catheter, or the like. For example, when the implant piece is a cube, the length (L) of the incision wound is, without limitation, shorter than the representative length (d) of the implant material required for implantation (where V=d³, V=volume of implant material), preferably (L/d)<0.9, more preferably (L/d)<0.7, and particularly preferably (L/d)<0.5.

In this embodiment, the implant piece may be preformed or cut after molding. When adjusting the size and shape of the final implant according to the subject at a clinical site, or the like, the implant piece can be effectively used by cutting at the clinical site.

The implant of the invention maintains mechanical properties, non-bioabsorbable properties, and/or biocompatibility, or the like even when it is implanted or placed in a living body for a long period of time, and therefore may be semi-permanently implanted or fixed in the body of a subject. However, the lower limit of the period of implantation or fixation may be 2 days or more, 5 days or more, 1 week or more, 1 month or more, 6 months or more, 1 year or more, 3 years or more, 5 years or more, 8 years or more, 10 years or more, or 15 years or more, and is preferably 5 years or more. The upper limit may be 50 years or less, 30 years or less, 15 years or less, 10 years or less, 6 years or less, 2 years or less, 10 months or less, or 5 months or less, and is preferably 30 years or less. The upper limit and the lower limit can be arbitrarily combined.

The subject may be healthy (for example, not have a specific or arbitrary disease) or suffer from any disease. When a treatment of a disease or the like is intended, it typically means a subject who suffers from a disease or is at risk of suffering from a disease. The subject is not limited as long as it is a mammal, but is preferably a human from the viewpoint that high biocompatibility has been confirmed in long-term implantation or placement. Therefore, in one embodiment, the invention is directed to an implant for humans.

In the present disclosure, the “treatment” includes all types of medically acceptable prophylactic and/or therapeutic interventions aimed at cure, temporary remission, prevention, or the like of a disease. For example, the “treatment” includes medically acceptable interventions for various purposes including delaying or stopping the progression of a disease, regression or disappearance of a lesion, prevention of the onset of the disease or prevention of recurrence of the disease, and the like.

The specific disease is not particularly limited as long as implantation or placement of an implant is required, but it can be suitably used for a disease involving long-term implantation or placement. For example, as the disease that requires long-term implantation, it is preferably used for a congenital or acquired defect in a body part, particularly, progressive hemifacial atrophy, breast cancer with breast resection (breast implant), pelvic cancer with radiation therapy (a sheet for preventing adhesion between organs or the like), hydrocephalus (VP shunt), or the like, and for example, as the disease that requires long-term placement, it is preferably used for urethral stenosis (urethra or bladder catheter) or the like, but it is not limited thereto.

The amide group-containing polymer compound that forms the nanocomposite hydrogel in the invention is obtained by polymerizing an amide group-containing monomer that dissolves in water, and the obtained amide group-containing polymer compound dissolves or swells in water at a biological temperature of 35 to 37° C. In addition, the amide group-containing polymer compound may be a polymer compound also having a functional group with an affinity for water (for example, an ester group, an ether group, an amino group, a carboxylic acid group, a sulfonic acid group, a hydroxy group, or the like) in addition to an amide group.

As a specific example of the amide group-containing polymer compound, a polymer compound obtained by polymerizing one type or two or more types selected from amide group-containing polymerizable monomers such as N-alkylacrylamide, N,N-dialkylacrylamide, N-alkylmethacrylamide, N,N-dialkylmethacrylamide, and acrylamide is exemplified.

Specific examples of the amide group-containing polymer compound that dissolves in water at a biological temperature (35 to 37° C.) include poly(N-methylacrylamide), poly(N-ethylacrylamide), poly(N-ethylmethacrylamide), poly(N-cyclopropylacrylamide), poly(methacrylamide), poly(N-methylmethacrylamide), poly(N-cyclopropylmethacrylamide), poly(N-cyclopropylmethacrylamide), poly(N-isopropylmethacrylamide), poly(N, N-dimethylacrylamide), poly(N-methyl-N-ethylacrylamide), poly(N-acryloylpyrrolidine), poly(N-acryloylmorpholine), poly(N-acryloylmethylpiperazine), and poly(acrylamide). Among these, one having no critical temperature indicating a phase transition is particularly preferably used.

Further, the combined use of the above-mentioned amide group-containing polymerizable monomer with another polymerizable monomer (for example, a nonionic water-soluble monomer, a cationic water-soluble monomer, an anionic water-soluble monomer, or the like) is also possible as long as the physical properties and function of the nanocomposite hydrogel according to the invention are maintained. In addition, a stimulus-responsive water-soluble polymer which has a critical temperature indicating a phase transition at a temperature lower than the biological temperature and changes the gel volume by an external stimulus such as poly(N-isopropylacrylamide), poly(N,N-diethylacrylamide), or poly(N-methyl-N-isopropylacrylamide) is also used when it is modified to dissolve or swell in water at 35 to 37° C. by copolymerization or the like.

The inorganic clay that forms the nanocomposite hydrogel used in the invention is preferably a water-swellable inorganic clay that has a charge on the surface of a clay layer and swells or peels off in layers in water, and more preferably one that is finely dispersed in water in a small unit of a single layer (with a thickness of about 1 nm) or multiple layers to form an inorganic clay nanosheet. Specific examples of a preferred inorganic clay include water-swellable hectorite containing sodium or the like as interlayer ions, water-swellable montmorillonite, water-swellable saponite, and water-swellable synthetic mica. In addition, one in which some or all of the hydroxy groups of such an inorganic clay are fluorinated is also used as long as it is dispersed in water to form an inorganic clay nanosheet. More preferred is a synthetic inorganic clay, with which the size of a nanosheet after peeling off in layers is small, and the diameter thereof is 10 to 500 nm, more preferably 20 to 300 nm, and particularly preferably 20 to 100 nm. In this case, an aqueous dispersion liquid of an inorganic clay nanosheet having peeled off in a single layer is easily obtained by stirring in water.

The amounts of the amide group-containing polymer compound and the inorganic clay that form the nanocomposite hydrogel used in the invention are preferably such that the amide group-containing polymer compound and the inorganic clay nanosheet form a three-dimensional network (organic-inorganic network) and mechanical properties including flexibility and toughness are exhibited. Specifically, the mass ratio of the inorganic clay/the amide group-containing polymer compound is preferably 0.1 to 1.8, more preferably 0.15 to 1.3, even more preferably 0.2 to 1.0, and particularly preferably 0.2 to 0.8. When the mass ratio is 0.1 or less, the softness is excellent, but the mechanical toughness may often be poor, and when the mass ratio is 1.8 or more, the softness may sometimes be poor.

The nanocomposite hydrogel used in the invention contains a solvent in the three-dimensional network. As the solvent, water or an aqueous solution containing a solute useful in vivo (for example, physiological saline) is used. The mass ratio of water/the solid content in a nanocomposite-type hydrogel is not necessarily limited as long as the mechanical properties of the nanocomposite hydrogel required in the invention are maintained, but is preferably 3 to 300, more preferably 5 to 50, and particularly preferably 6 to 20. When the mass ratio is 3 or less, the softness may sometimes be poor, and when the mass ratio is 300 or more, the mechanical toughness may sometimes be poor.

In the nanocomposite hydrogel in the invention, it is necessary to form an organic (polymer)-inorganic (clay nanosheet) network structure using the inorganic clay nanosheet having peeled off in layers in water as a polyfunctional crosslinking agent for the amide group-containing polymer compound (that is, a large number of amide group-containing polymer compounds are crosslinked by the inorganic clay nanosheet). When the inorganic clay does not sufficiently peel off in layers in water to form a nanosheet or when a large number of polymer compound chains are not bonded (surface-crosslinked) to each inorganic clay nanosheet, the nanocomposite hydrogel effective in the invention is not formed.

On the other hand, in order to control mechanical properties or precisely suppress excessive swelling, or to suppress a change in shape or physical properties due to a sterilization treatment by an autoclave, the combined use of crosslinking (chemical crosslinking) with a small amount of an organic crosslinking agent together with a water-swellable clay mineral is effective. Examples of such an organic crosslinking agent include difunctional compounds such as N,N′-methylenebisacrylamide, N,N′-propylenebisacrylamide, di(acrylamidemethyl)ether, 1,2-diacrylamide ethylene glycol, 1,3-diacryloyl ethylene urea, ethylene glycol diacrylate, ethylene glycol dimethacrylate, N,N′-diallyl tartardiamide, and N,N′-bisacrylyl cystamine, and trifunctional compounds such as triallyl cyanurate and triallyl isocyanurate.

Further, the amount of the organic crosslinking agent to be used needs to be small, and preferably, the ratio of the organic crosslinking agent to the repeating unit of the amide group-containing polymer compound is preferably 0.001 to 0.5 mol %, more preferably 0.005 to 0.3 mol %, and particularly preferably 0.01 to 0.2 mol %. When the ratio of the organic crosslinking agent is 0.001 mol % or less, the effect of using the organic crosslinking agent is small, and when the ratio is 0.5 mol % or more, the mechanical toughness may sometimes not be sufficient. Further, in the nanocomposite hydrogel in the invention, in order to further control mechanical properties, or control swellability, or control density, hardness, and further biocompatibility, or the like, incorporation of another organic polymer compound or an inorganic component in the nanocomposite hydrogel is effectively used. Examples of the organic polymer compound include polyethylene glycol, polytetrafluoroethylene, polyvinyl alcohol, and collagen, and examples of the inorganic component include hydroxyapatite, silica, and titania.

As a method for producing the nanocomposite hydrogel in the invention, it can be synthesized by a conventionally reported method. Specifically, for example, a method for producing a nanocomposite hydrogel, in which dissolved oxygen is removed from a uniform aqueous solution formed of an amide group-containing monomer and an inorganic clay nanosheet, or a uniform aqueous solution formed by adding a water-soluble monomer having a functional group other than an amide group or an organic crosslinking agent thereto, and then, a polymerization initiator and, if necessary, further a catalyst are dissolved or uniformly dispersed therein, and then, the contained monomers (and the organic crosslinking agent) are polymerized, is used.

As the polymerization initiator, a thermal polymerization initiator (for example, a peroxide) is used, and also a photopolymerization initiator can be used. In the latter case, the polymerization can be started by UV irradiation. Specifically, as the thermal polymerization initiator, a water-soluble peroxide, for example, potassium peroxodisulfate or ammonium peroxodisulfate, a water-soluble azo compound, or the like can be preferably used. For example, VA-044, V-50, or V-501 manufactured by Wako Pure Chemical Industries, Ltd. or the like can be used. In addition, a water-soluble radical initiator having a polyethylene oxide chain or the like is also used. Further, when polymerization is performed by UV irradiation, a hydrophilic or hydrophobic photopolymerization initiator is used. Specific examples of the photopolymerization initiator include acetophenones such as p-tert-butyltrichloroacetophenone, benzophenones such as 4,4′-bisdimethylaminobenzophenone, ketones such as 2-methylthioxanthone, benzoin ethers such as benzoin methyl ether, α-hydroxyketones such as hydroxycyclohexylphenylketone, phenylglyoxylates such as methylbenzoylformate, and metallocenes.

Further, as the catalyst, N,N,N′,N′-tetramethylethylenediamine or β-dimethylaminopropionitrile, each of which is a tertiary amine compound, or the like is preferably used. The polymerization temperature is set within the range of 0° C. to 100° C. according to the types of water-soluble organic monomer, polymerization catalyst, and initiator to be used, or the like. The polymerization time also varies depending on the polymerization conditions such as the catalyst, the initiator, the polymerization temperature, and the amount of the polymerization solution, and although it cannot be unconditionally specified, it is generally performed for a time between several tens of seconds and several tens of hours.

As a result of the above, the nanocomposite hydrogel in which the amide group-containing polymer compound and the inorganic clay nanosheet having peeled off in layers are combined to form a three-dimensional network (organic-inorganic network), or the nanocomposite hydrogel in which along with the formation of such an organic-inorganic network, a composite network is formed by further binding the amide group-containing polymer compound around the inorganic clay nanosheet with a small amount of a chemical crosslink is obtained. When the composite network is formed, flexibility and water swellability are controlled, and in addition, particularly, it has a characteristic such that changes in shape and mechanical properties of the nanocomposite hydrogel caused by a sterilization treatment with an autoclave (121° C., 2 atm, 30 minutes) are suppressed.

In one embodiment, the nanocomposite hydrogel used in the invention has an elastic modulus in the range of 1 to 1000 kPa, and the elongation and the tensile strength at rupture are 150% or more and 20 kPa or more, respectively.

Those skilled in the art can select the required elastic modulus, elongation ratio, and strength at rupture according to the type of implant using the nanocomposite hydrogel, the position, the method, etc. For example, when the nanocomposite hydrogel is used as a soft implant material, the elastic modulus is preferably in the range of 1 to 600 kPa, and the elongation and the tensile strength at rupture are preferably 200% or more and 30 kPa or more, respectively, more preferably 400% or more and 50 kPa or more, respectively, and particularly preferably 500% or more and 80 kPa or more, respectively. The elastic modulus, the elongation, and the tensile strength at rupture of the nanocomposite hydrogel can be measured using a tensile test apparatus under the conditions of, for example, a distance between marks of 30 mm, a tensile speed of 100 mm/min, and a temperature of 25° C.

The nanocomposite hydrogel used in the invention has the elastic modulus, the elongation ratio, and the strength at rupture as described above, and in one embodiment, also has compression resistance and mechanical toughness. As the compression resistance, it has toughness so that it does not rupture by preferably 85% compression, more preferably 90% compression, and particularly preferably 95% compression. Further, as the mechanical toughness, it is one having folding resistance and torsional resistance, specifically, one which is not ruptured by a 180-degree fold, and also is not ruptured by preferably a 180-degree twist, more preferably a 270-degree twist, and particularly preferably a 360-degree twist. By using such a nanocomposite hydrogel, it can be handled without worrying about rupture at the time of operation, and it can be easily handled using various instruments to be used at a clinical site. As for the folding resistance and torsional resistance, the occurrence of a crack or rupture can be visually confirmed using a conventional method.

The nanocomposite hydrogel used in the invention is preferably optically transparent. Specifically, the light transmittance in the thickness direction at a wavelength of 600 nm measured by fixing the hydrogel having a thickness of 10 mm to an ultraviolet-visible spectrophotometer is preferably 50% or more, more preferably 70% or more, and particularly preferably 90% or more.

The nanocomposite hydrogel used in the invention can be sterilized by high-pressure steam sterilization (autoclave) in one embodiment. The sterilization by an autoclave is not particularly limited as long as it is performed under the conditions used for a sterilization treatment in this technical field, but the conditions can be arbitrarily selected from the ranges of 110° C. to 140° C., 1.2 to 10 atm, 3 to 30 minutes, and the like. Therefore, the implant containing such a nanocomposite gel can be sterilized in the same manner as other surgical instruments or the like, and infection can be easily prevented.

As the nanocomposite hydrogel used in the invention, one having a wide range of size and a simple to complex shape, and also having a simple to complex surface morphology can be prepared by changing the shape and size of a reaction container or a template used in the synthesis process. As the reaction container, it is effective to use a resin film or a resin tube having low oxygen permeability in order to improve productivity, in addition to a container made of a glass or a plastic having a general smooth surface or various uneven surfaces. As a result, the nanocomposite hydrogel having any size, shape, and surface morphology according to a case or larger than that is obtained.

In one embodiment, the nanocomposite hydrogel used in the invention can maintain the mechanical properties even when it is implanted or placed for a long period of time. Here, the maintenance of the mechanical properties means, for example, that a change in one or more parameters indicating mechanical properties selected from the group consisting of an elastic modulus, an elongation, a tensile strength at rupture, compression resistance, and mechanical toughness before and after implantation or placement for 1 year or more, 3 years or more, 5 years or more, 7 years or more, 9 years or more, or 15 years or more is 50% or less, 40% or less, 30% or less, 20% or less, 10% or less, 5% or less, 3% or less, 1% or less, or 0.5% or less, preferably 20% or less throughout the implantation for 9 years or more, and more preferably, the parameters do not change. Therefore, even when the implant containing such a nanocomposite gel is implanted or placed in a subject, the nanocomposite gel is not ruptured or damaged, so that the implant can function stably for a long period of time.

In one embodiment, the nanocomposite hydrogel used in the invention can maintain non-bioabsorbable properties even when it is implanted or placed for a long period of time. The maintenance of the non-bioabsorbable properties means, for example, that a decrease in solid mass of the nanocomposite hydrogel before and after implantation or placement for 1 year or more, 3 years or more, 5 years or more, 7 years or more, 9 years or more, or 15 years or more is 20% or less, 10% or less, 5% or less, 3% or less, 1% or 0.5% or less, preferably the decrease is 2% or less throughout the implantation for 9 years or more, and more preferably, the decrease is 1% or less. Therefore, even when the implant containing such a nanocomposite gel is implanted or placed, the nanocomposite gel component is not decomposed or eluted in a living body, so that it is non-toxic and can be safely used for a long period of time.

In one embodiment, the nanocomposite hydrogel used in the invention can maintain biocompatibility even when it is implanted or placed in a subject for a long period of time. The “biocompatibility” in the invention refers to a property in which cytotoxicity, a foreign body reaction, and/or adhesion between a living body and the nanocomposite hydrogel does not occur.

The maintenance of the biocompatibility means that in a body part where the nanocomposite hydrogel is implanted or placed, one or more parameters selected from the group consisting of an increase in the number of cells such as leukocytes (neutrophils, lymphocytes, etc.) or macrophages, a marker elevation of an inflammatory marker such as CRP, MMP-3, or an inflammatory cytokine, a manifestation of pathology such as edema, exudation, or necrosis of a connective tissue around a blood vessel, a manifestation of a symptom caused by a chronic foreign body reaction such as fibril formation or capsular contracture, an adhesion reaction between the nanocomposite hydrogel and a living body, and vascular invasion into the nanocomposite hydrogel caused by the nanocomposite hydrogel are not observed throughout the implantation or placement for 1 year or more, 3 years or more, 5 years or more, 7 years or more, 9 years or more, or 15 years or more, preferably throughout the implantation for 9 years or more. Such a foreign body reaction can be confirmed by measurement of a conventional inflammatory marker, HE staining, immunostaining, visual or microscopic observation of the gel surface, or the like. Therefore, even when the implant containing such a nanocomposite gel is implanted or placed, a foreign body reaction or adhesion does not occur in the nanocomposite gel, so that the implant can be made to function safely for a long period of time and thereafter can also be easily removed.

In one embodiment, even when the nanocomposite hydrogel used in the invention is implanted or placed for a long period of time, the nanocomposite hydrogel itself can maintain non-bioabsorbable properties and/or biocompatibility. Therefore, even when the implant of the invention is cut into an arbitrary shape and implanted in a state where the cut surface is exposed, the same biocompatibility as before cutting can be maintained. For example, the nanocomposite hydrogel can be cut to a desired size, shape, and surface morphology using any instrument, for example, a general medical instrument (for example, a surgical knife, forceps, scissors, a twin hook, a single hook, a gimlet, tweezers, a drill, or the like). The naming such as shredding, crushing, drilling, or machining may differ depending on the size, shape, and surface morphology resulting from cutting the nanocomposite hydrogel, but these are all included in “cutting” in the present disclosure.

In an embodiment in which the implant is formed of the nanocomposite hydrogel, the cutting may be cutting of any part of the implant, and in an embodiment in which the implant contains the nanocomposite hydrogel, the cutting may be cutting of the part of the nanocomposite hydrogel to be used for the implant. The cutting may be performed at any time after the preparation of the nanocomposite hydrogel, but cutting at a clinical site is preferred from the viewpoint of requiring adjustment according to the state during an operation.

The nanocomposite hydrogel used in the invention can be adhered and bonded. The bonding does not require a general adhesive, and therefore, multiple nanocomposite hydrogels can be bonded while maintaining the functions such as the mechanical properties, non-bioabsorbable properties, and/or biocompatibility of the nanocomposite hydrogel used in the invention. Therefore, for example, by cutting the nanocomposite hydrogel in a rod shape or a hollow shape at a clinical site before implantation, and/or by adhering and bonding the cut surfaces, the nanocomposite hydrogel having a necessary shape and size can be processed and suitably used for the implant.

### EXAMPLES

Next, the invention will be more specifically described with reference to Examples, but the invention is of course not limited only to the Examples shown below.

### Reference Example 1

As a clay mineral, a water-swellable synthetic hectorite (trademark: Laponite-XLG, manufactured by Rockwood Ltd.) having the following composition [Mg_{5.34}Li_{0.66}Si₈O₂₀(OH)₄]Na⁺_{0.66} was used. As an organic monomer, N,N-dimethylacrylamide (DMAA, manufactured by Kohjin Co., Ltd.) was used after purification.

As a polymerization initiator, potassium peroxodisulfate (KPS, manufactured by Kanto Chemical Co., Inc.) was dissolved in deoxidized pure water at a ratio of KPS/water=0.20/10 (g/g) and used in the form of an aqueous solution. As a catalyst, N,N,N′,N′-tetramethylethylenediamine (TEM ED, manufactured by Kanto Chemical Co., Inc.) was used. All pure water was used after bubbling high-purity nitrogen for 3 hours or more in advance to remove oxygen contained therein.

In a constant temperature chamber at 20° C., 190 g of pure water from which dissolved oxygen was removed and a stirring bar made of polyethylene fluoride were placed in a flat-bottomed glass container having an inner diameter of 50 mm and a height of 150 mm, the inside of which was replaced with nitrogen, and 7.62 g of Laponite-XLG was added in small portions under stirring while being careful not to introduce air bubbles. The container was placed in a constant temperature water bath at 35° C. and heated for 20 minutes while stirring, and then, stirring was performed for 24 hours in a constant temperature chamber at 20° C., whereby a colorless transparent aqueous solution was prepared.

After cooling this aqueous solution with ice, 19.8 g of DMAA was added thereto, and the mixture was stirred for 5 minutes while bubbling nitrogen gas into the solution, whereby a colorless transparent solution was obtained. Thereafter, 10.0 g of a KPS aqueous solution and 160 μL of TEMED were added thereto while stirring, and the mixture was further stirred for about 15 seconds, whereby a uniform transparent reaction solution was obtained. A portion of this reaction solution was filled in a film forming container with a closed bottom having a thickness of 2 mm and a width and a length of 150 mm (the inside is a resin film laminate (transparent vapor-deposited stretched polyethylene terephthalate 12 μm/stretched nylon 15 μm/unstretched polypropylene 60 μm) bag) without contact with oxygen, and thereafter, the inlet was tightly sealed and the container was allowed to stand for 12 hours in a constant temperature water bath at 50° C. for polymerization. A portion of the solution (43 g×3) was transferred to three glass cylindrical gel forming containers having an inner diameter of 36.5 mm and a height of 60 mm without contact with oxygen, and thereafter, the inlet was tightly sealed and the containers were allowed to stand for 12 hours in a constant temperature water bath at 50° C. for polymerization.

All the procedures from the preparation of these solutions to the polymerization were performed in a nitrogen atmosphere in which oxygen was blocked. After the polymerization reaction, a uniform transparent hydrogel having elasticity and flexibility was taken out from each of the film gel forming container and the cylindrical gel forming container. After taking out the hydrogel, the surface was lightly washed with pure water. In the hydrogel, the entire solution used for the polymerization was gelled to form uniform transparent film-like and cylindrical hydrogels, and no non-uniform aggregation due to the inorganic clay or the polymer was observed in the hydrogel. The fact that the component of this hydrogel is formed of poly(N,N-dimethylacrylamide) (PDMAA), the inorganic clay, and water was confirmed from Fourier transform infrared absorption spectrum measurement (using a Fourier transform infrared spectrophotometer FT/IR-550, manufactured by JASCO Corporation) by the KBr method using a dried material of this hydrogel.

Further, in the composition of the hydrogel, the mass ratio of water/the solid content (inorganic clay+PDMAA) is 7.3 and the mass ratio of the inorganic clay/PDMAA is 0.38, the former was revealed by drying the hydrogel to a constant mass with a vacuum dryer at 120° C., and the latter was revealed by a thermal mass analysis of the dried hydrogel material (TG-DTA220 manufactured by Seiko Instruments & Electronics, Ltd., heated to 600° C. at 10° C./min under air flow). Further, the dried hydrogel material was embedded in an epoxy resin, and an ultrathin section was prepared with an ultramicrotome and measured with a high-resolution electron microscope (JEM-200CX manufactured by JEOL Ltd.) at an acceleration voltage of 100 KV. As a result, it was confirmed that the inorganic clay nanosheet having peeled off in one to two layers or so was uniformly dispersed in a polymer matrix.

The obtained hydrogel could be processed into various shapes using a surgical knife, forceps, scissors, a single hook, tweezers, or the like without causing cracks or the like. For example, the film-like hydrogel was cut into a strip shape having a width of 10 mm and a length of 70 mm (a thickness of 2 mm), and the following experiments were performed. One was subjected to torsional deformation of 180 degrees, 360 degrees, and two turns (2×360 degrees) in the vertical direction. Further, one was folded once and three times. As a result, in each case, the hydrogel did not crack or rupture, and reversible deformation was possible. In addition, the same strip-shaped hydrogel was attached to a tensile test apparatus (manufactured by Shimadzu Corporation, a desktop universal testing machine AGS-H) without slipping on the chuck, and a tensile test was performed by setting the distance between marks to 30 mm and the tensile speed to 100 mm/min. As a result, the tensile strength was 165 kPa, the elongation at rupture was 1450%, and the elastic modulus was 8.0 kPa. Further, a hydrogel having a thickness of 10 mm cut out from the cylindrical hydrogel was fixed in a UV-visible spectrophotometer (V-530, manufactured by JASCO Corporation), and the light transmittance in the thickness direction of the hydrogel was measured. As a result, it was confirmed that the light transmittance was 95% at a wavelength of 600 nm.

In addition, a cubic hydrogel having a side of 10 mm was cut out from the cylindrical hydrogel, and a test of compression by 80% and 95% in the thickness direction was performed. As a result, reversible compressive deformation was possible without cracks or rupture due to the occurrence of a defect in the hydrogel in all cases.

From the above results, it was confirmed that the obtained hydrogel is a nanocomposite hydrogel that is obtained through formation of an organic-inorganic network structure in which an inorganic clay nanosheet and poly(N,N-dimethylacrylamide) which is an amide group-containing polymer compound are linked, and that is uniform and transparent and has excellent mechanical properties (high elongation and mechanical toughness to withstand compression, bending, twisting, or the like).

### Example 1

### <High-Pressure Steam Sterilization>

The film-like hydrogel (nanocomposite hydrogel) prepared in Reference Example 1 was cut to obtain two sheets with a size of 70 mm×70 mm×2 mm (thickness). Further, the cylindrical hydrogel (nanocomposite hydrogel) prepared in Reference Example 1 was cut at both ends to form a cylindrical shape having a diameter of 36.5 mm and a length of 35 mm. Each of these was placed in an autoclave sterilization bag and set in an autoclave apparatus (apparatus name: autoclave SX-700, manufactured by Tomy Seiko Co., Ltd.), and high-pressure steam sterilization was performed. The treatment conditions were set to 121° C. and 20 minutes. In addition, in the sterilization bag including one sheet of the film-like hydrogel, a biological indicator product (manufactured by Raven Biological Laboratories, Inc., hereinafter referred to as BI) containing spores of Bacillus stearothermophilus, which is an indicator microorganism of sterilization, was enclosed together with the hydrogel. After the treatment, the sterilized hydrogel included in the sterilization bag was taken out from the apparatus, and the viable microorganisms in BI were confirmed. As a result, it was confirmed that the microorganisms had died. In addition, there was no significant change in the shape of the film-like hydrogel after sterilization. On the other hand, there was also no significant change in the cylindrical hydrogel except that the cylindrical shape slightly changed to an elliptical cylindrical shape.

### <Cytotoxicity Test>

In addition, an in vitro cytotoxicity test was performed using the obtained sterilized film-like and cylindrical hydrogels according to ISO 10993-5 (1999). A sample solution was obtained by extraction from the hydrogel with Eagle's minimum essential medium at 37° C. for 24 hours. V79 cells (JCRB cell bank, Japan) were cultured in a medium to which the sample extract solution of a different concentration (0 to 100%) was added at 37° C. for 7 days in a 5% CO2 atmosphere. Cytotoxicity was quantitatively evaluated by counting the number of living colonies after cell culture with a colony counter ProtoCOL System (Synoptics Ltd., Japan) for each sample. As a result, regardless of the extract solution concentration, the number of colonies was 90% or more of that without adding the sample extract solution, and it was confirmed that there is no cytotoxicity.

Each of a material obtained by cutting the obtained film-like hydrogel to a diameter of 70 mm and a material obtained by cutting the obtained cylindrical hydrogel to a side length of 30 mm and a thickness of 5 mm was transferred into a cell culture dish (“Falcon 3003” manufactured by Becton Dickinson Labware) in a clean bench, and the dish was covered and allowed to stand at 37° C. to culture the cells. As the cells to be cultured, normal human skin fibroblasts (manufactured by Dainippon Pharmaceutical Co., Ltd.) were used. The culture was performed using CS-C medium (manufactured by Dainippon Pharmaceutical Co., Ltd.) in an incubator at 37° C. containing 5% carbon dioxide. One week after inoculation, the dish was allowed to stand for 5 minutes in a constant temperature bath at 20° C., and then the surface was observed with an optical microscope. As a result, it was confirmed that no cells adhered to any of the hydrogels.

### <Implantation Test in Human>

### Example 2

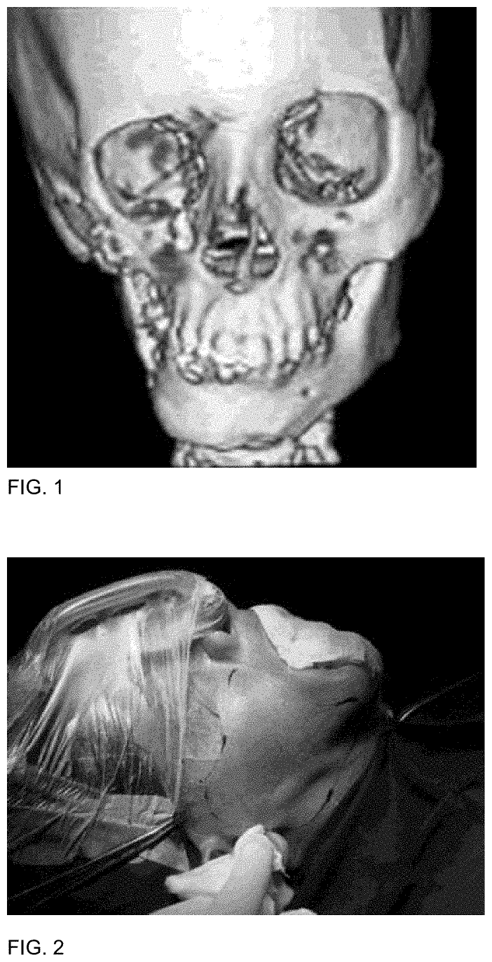

Below, implantation surgery was performed on a subject (13 years old, female) with progressive hemifacial atrophy (Parry Romberg syndrome), and a 9-year survey was conducted. As the physical finding of the subject, facial asymmetry due to marked hypoplasia on the right side of the face, deviation of the lips and nose to the right side, slight enophthalmos of the right eye, etc. was observed. Further, the anterior teeth on the right side were exposed due to the notch and thinning of the upper and lower lips. In addition, excessive pigmentation was observed in the skin of an upper part of the malar arch. FIG. 1 shows a CT image of the subject. It can be seen that the cheekbone of the subject is missing.

As a treatment method, it was determined that first, an implant formed of a nanocomposite hydrogel (hereinafter, also referred to simply as “implant” or “implant piece”) is implanted, and then, mandibular extension surgery is performed after a predetermined period of time. This treatment was performed in accordance with the Declaration of Helsinki.

First, an implant implantation planned region of the subject, that is, the circumference of the cheek region was marked (see FIG. 2). In order to avoid leaving a scar in the face of the subject or damaging the facial nerve when implanting in the cheek region, two small incision wounds were made in wrinkled parts of the skin in a side part of the auricle and a lower part of the orbit of the subject, and the SMAS layer was subjected to flap. The incision sites were carefully selected so that the skin would not contract or adhere.

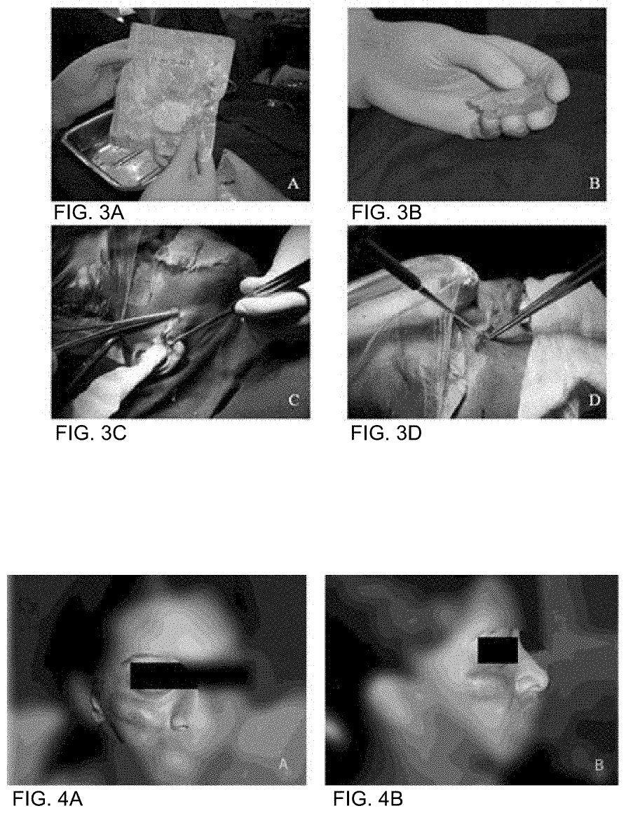

In order to implant the implant in the cheekbone region through the incision wounds, a portion was cut out from the cylindrical nanocomposite hydrogel prepared in Reference Example 1 and previously sterilized in Example 1 (see the photograph in FIG. 3B), which was further cut into several thin pear-shaped hydrogels with a size of about 20 mm to 40 mm×about 30 mm to 60 mm×2 to 4 mm with a surgical knife. The thus shredded implants were inserted in the cheekbone region one piece at a time multiple times through the incision wounds (each length: 5 mm) in the lower part of the orbit and under the auricle so as to reconstruct the soft tissue of the cheekbone region (the total mass of the implanted gel was 8.7 g, the calculated solid mass=1.05 g). Note that the shredded implants were not particularly subjected to a coating treatment or the like. The manner in which the shredded implants were inserted one piece at a time with tweezers through the lower part of the auricle and the lower part of the orbit is shown in FIGS. 3C and 3D.

No special fixing treatment such as suturing or gluing the implant to the cheek region was performed. Thereafter, the incision wounds were sutured with an absorbable thread. The surface of the lower part of the orbit was fixed with a skin adhesive tape (manufactured by 3M) (in order to avoid postoperative edema). After the operation, there was slight swelling for about 1 week, but it subsided in about 10 days. The length (L) of the incision wound at the time of implantation with respect to the representative length (d) obtained from the volume of the hydrogel used was L/d=0.25.

The appearance of the subject one year after the operation is shown in FIGS. 4A and 4B. It can be seen that there is a soft tissue-like swelling supplemented by the implant in the right cheek region of the subject. The implant did not move from when the operation was performed despite the daily movement of the face, and further, there was no change in the color and hardness of the skin as well as the morphology such as shape and size in appearance.

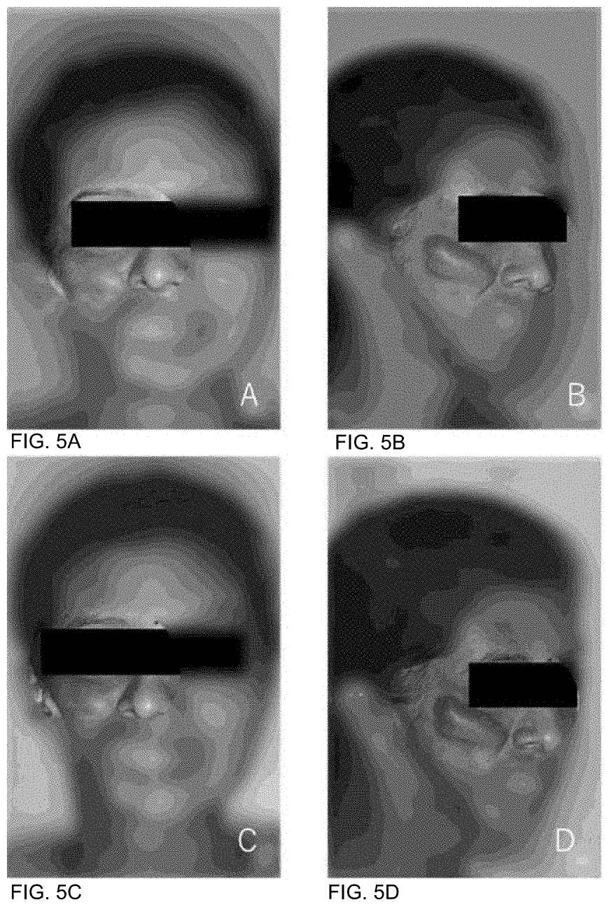

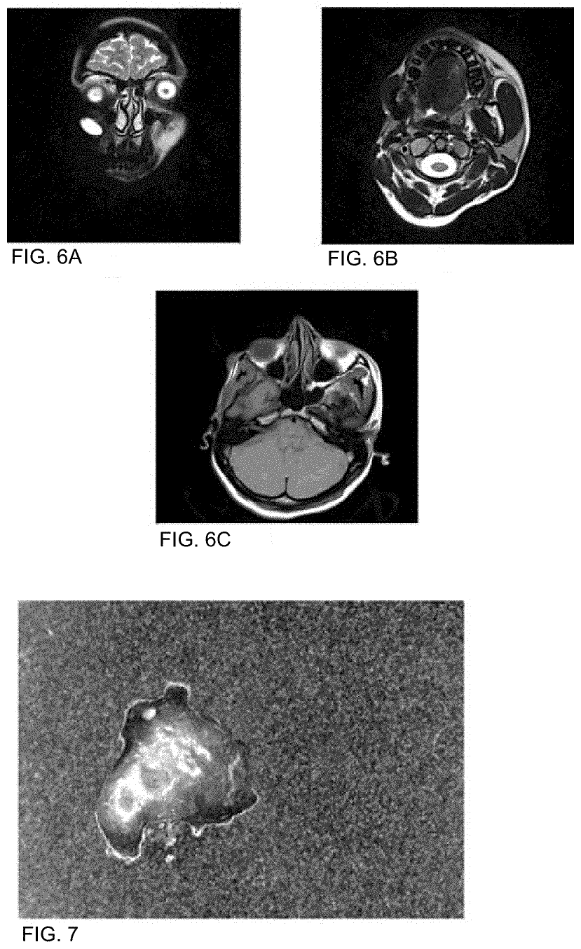

An examination was performed again 7 and 9 years after the operation. As a result of the examination, the implanted implant did not move from when the operation was performed, and the structure such as shape and size in appearance was maintained, and no abnormality was observed in the surrounding tissue. In addition, there was no change in the color and hardness of the skin. FIGS. 5A-5D show the appearance of the subject 7 years after the operation (FIGS. 5A and % B) and 9 years after the operation (FIGS. 5C and 5D). It can be seen that the disease has slightly progressed, but there is no change in appearance in the implant and the surrounding tissue. In addition, also from the MRI images in FIGS. 6A-6C, taken 7 years after the operation, it can be seen that no foreign body reaction such as inflammation or capsular contracture has occurred in and around the implant implantation region. Further, it was confirmed that the implant was stably present in the same manner also in the MRI images 9 years after the operation, and that no foreign body reaction has occurred in the implantation region and the surrounding tissue.

On the other hand, with the progression of Parry Romberg syndrome and the growth of the subject, a strong asymmetry occurred in the face of the subject. Therefore, at the age of 22 years, 9 years after the operation (13 years old), the implanted implant was removed from the cheek region, and thereafter, the entire face was reinforced by fat transplantation.

Therefore, a small incision wound was made in a side part of the auricle (about 9 mm long), followed by flap, and the implant that had been implanted for 9 years was taken out. The total amount of each implant piece could be easily taken out without adhesion to the surrounding tissue (total mass=9.1 g, (dry) solid mass=1.05 g). The length (L) of the incision wound at the time of taking out was L/d=0.45.

The nanocomposite hydrogel taken out had substantially the same transparency and shape as when it was inserted, and there was no adhesion of biological tissues around and inside the hydrogel, and no invasion of blood vessels or the like into the hydrogel was confirmed. When a histopathological examination of a tissue surrounding the implantation site was performed, it was confirmed that there is no aggregation of foreign body giant cells, granulomas, fibril formation, sign associated with a foreign body reaction such as capsular contracture, or infection. An optical microscopic image of the implant piece taken out is shown in FIG. 7. It can be seen that the implant piece has the same transparency as before implantation, and there is no adhesion of a tissue piece or the like, vascular invasion, or the like. Further, one of the implant pieces taken out was subjected to torsional deformation of 180 degrees, 360 degrees, and two turns (2×360 degrees) in the vertical direction. In addition, one was folded once and three times. As a result, in each case, the hydrogel did not crack or rupture, and the same reversible deformation as before implantation was possible. It was confirmed that the nanocomposite hydrogel maintained excellent mechanical properties for a long period of time as an implant.

The mass (solid mass) after drying of the total amount of the nanocomposite hydrogel taken out was 1.04 g. Since there was no change in the solid mass before and after implantation, it was confirmed that no gel fragment was missing from the nanocomposite hydrogel even in long-term implantation. Further, from the FTIR observation of the dried material, it was confirmed that there is no change in the spectra of PDMAA, which is a polymer component of the hydrogel used, and hectorite, which is an inorganic component. In addition, no trace of calcium deposition or the like was observed, and it was confirmed that the nanocomposite hydrogel maintained excellent biocompatibility over a long period of time (9 years) as an implant without being absorbed into the body, and also without causing any foreign body reaction or adhesion.

### Comparative Example 1

A chemically crosslinked PDMAA hydrogel was prepared by the same synthetic method as in Example 1 except that an organic crosslinking agent (N,N′-methylenebisacrylamide) was used in an amount of 2 mol % with respect to DMAA in place of the inorganic clay. The obtained hydrogel was brittle and ruptured when it was tried to be taken out from the film container, and could not be taken out in a predetermined shape. In addition, a portion thereof taken out ruptured in a 90-degree twist test, and could not be folded even once and ruptured. Therefore, the obtained chemically crosslinked hydrogel could not be used as an implant material.

Those skilled in the art will understand that many various modifications can be made without departing from the spirit of the present invention. Therefore, it should be understood that the embodiments of the invention described herein are merely exemplary and are not intended to limit the scope of the invention.

1. An implant, characterized by comprising a nanocomposite hydrogel obtained through formation of an organic-inorganic network structure in which an inorganic clay nanosheet and an amide group-containing polymer compound are linked.

2. The implant according to claim 1, characterized by being implanted or placed in a human living body.

3. The implant according to claim 1, characterized in that the mass ratio of the inorganic clay nanosheet to the amide group-containing polymer compound is in a range of 0.1 to 1.8.

4. The implant according to claim 1, characterized in that the amide group-containing polymer compound is a polymer obtained by polymerizing one or more polymerizable unsaturated group-containing water-soluble organic monomers having an amide group such as N-alkylacrylamide, N,N-dialkylacrylamide, acrylamide, N-alkylmethacrylamide, N,N-dialkylmethacrylamide, and methacrylamide.

5. The implant according to claim 1, characterized in that the amide group-containing polymer compound dissolves or swells in water at a biological temperature and is capable of forming a nanocomposite hydrogel having optical transparency at a biological temperature.

6. The implant according to claim 1, characterized by being implanted in a load region.

7. The implant according to claim 1, characterized by being used for prosthesis for a soft tissue.

8. The implant according to claim 7, characterized in that the soft tissue is a soft tissue of the face or the breast.

9. The implant according to claim 1, characterized by being able to be cut and/or bonded at a clinical site.

10. A method of treating a disease requiring implantation or placement of an implant in a subject, comprising applying the implant to the subject in need thereof,
wherein the implant comprises a nanocomposite hydrogel obtained through formation of an organic-inorganic network structure in which an inorganic clay nanosheet and an amide group-containing polymer compound are linked.
